(19) United States
(12) Patent Application Publication
(10) Pub. No.: US 2018/0078424 A1
(43) Pub. Date: Mar. 22, 2018

(54) PEEL AND STICK HEATING PAD FOR BREAST

(71) Applicant: Joseph J. Galazin, JR., Greenwich, CT (US)

(72) Inventor: Joseph J. Galazin, JR., Greenwich, CT (US)

(21) Appl. No.: 15/710,192

(22) Filed: Sep. 20, 2017

Related U.S. Application Data
(60) Provisional application No. 62/396,989, filed on Sep. 20, 2016.

(51) Int. Cl. A61F 13/14; A61F 7/02; A61F 7/03

(52) U.S. Cl. CPC A61F 13/14; A61F 7/0241; A61F 2007/0098; A61F 2007/0268; A61F 7/03

(57) ABSTRACT

An air activated therapeutic heating pad for breast is shown and described herein. The heating pad may include a plurality of pad members. Each of the plurality of pad members may include a facestock layer, a first pressure sensitive adhesive, a second pressure sensitive adhesive, and a liner layer. Each of the plurality of pad members may be capable of being selectably activated and the each of the plurality of pad members may comprise a heat-generating composition generating a heat to a temperature. The plurality of pad members may form a generally U-shape capable of fitting within a cup of a bra.

### CROSS-REFERENCE TO RELATED APPLICATIONS

The present application claims priority to and the benefit of U.S. Provisional Patent Application No. 62/396,989 entitled “PEEL AND STICK HEATED BREAST PAD” filed on Sep. 20, 2016 and is hereby incorporated by reference in its entirety.

### FIELD OF THE INVENTION

The present invention relates generally to a heating pad for a breast of a user and, more particularly, to a peel and stick pad capable of removably adhering to a breast and/or bra of a user and warming said breast.

### BACKGROUND

Warming or heating of a body part, especially a breast of a user, can have certain advantageous impact. Accordingly, there is a desire to utilize a heating device for such. An example would be a heating pad. However, utilizing a heating pad may require electrical power, such as a plug and outlet, which may make heating the breast impossible during certain portions of a user's day. As such, there is a need for a heating device that is portable and discrete enough to avoid detection. The present teachings overcome these shortcomings and provide a heating device for a breast of a user that is portable and discrete.

### SUMMARY

An air activated therapeutic heating pad for breast is shown and described herein. The heating pad may include a plurality of pad members. Each of the plurality of pad members may include a facestock layer, a first pressure sensitive adhesive, a second pressure sensitive adhesive, and a liner layer. Each of the plurality of pad members may be capable of being selectably activated and the each of the plurality of pad members may comprise a heat-generating composition generating a heat to a temperature. The plurality of pad members may form a generally U-shape capable of fitting within a cup of a bra.

A method of manufacturing the heating pad may include providing a heat-generating material into the facestock layer. The method may also include applying a first pressure sensitive adhesive to a surface disposed on the facestock layer. A second pressure sensitive adhesive material may be applied to a surface disposed on the liner layer. The method further includes forming the material into the pad defining an enclosure.

Specific reference is made to the appended claims, drawings, and description below, all of which disclose elements of the invention. While specific embodiments are identified, it will be understood that elements from one described aspect may be combined with those from a separately identified aspect. In the same manner, a person of ordinary skill will have the requisite understanding of common processes, components, and methods, and this description is intended to encompass and disclose such common aspects even if they are not expressly identified herein.

### BRIEF DESCRIPTION OF THE DRAWINGS

The operation of the invention may be better understood by reference to the following detailed description taken in connection with the following illustrations, wherein:

FIG. 1 is a perspective view of an embodiment of a peel and stick heating pad for breast adhered to a bra;

FIG. 2 is a perspective view of an embodiment of a peel and stick heating pad for breast;

FIG. 3 is a side view of an embodiment of the peel and stick heating pad for breast;

FIG. 4 is a side view of another embodiment of the peel and stick heating pad for breast showing the heating elements;

FIG. 5 is a cross-sectional view of an embodiment of the peel and stick heating pad for breast before peeling off of a liner layer;

FIG. 6 is another cross-sectional view of an embodiment of the heating elements of the peel and stick heating pad for breast after peeling off of the liner layer;

FIG. 7 is a front view of a cross-sectional view of an embodiment of the peel and stick heating pad for breast;

FIG. 8 is another front view of an embodiment of the peel and stick heating pad for breast;

FIG. 9 is an exemplary view of another embodiment of the heating elements of the peel and stick heating pad for breast; and

FIG. 10 is another exemplary embodiment of the heating elements of the peel and stick heating pad for breast.

### DETAILED DESCRIPTION

Reference will now be made in detail to exemplary embodiments of the present invention, examples of which are illustrated in the accompanying drawings. It is to be understood that other embodiments may be utilized and structural and functional changes may be made without departing from the respective scope of the invention. Moreover, features of the various embodiments may be combined or altered without departing from the scope of the invention. As such, the following description is presented by way of illustration only and should not limit in any way the various alternatives and modifications that may be made to the illustrated embodiments and still be within the spirit and scope of the invention.

As used herein, the words “example” and “exemplary” mean an instance, or illustration. The words “example” or “exemplary” do not indicate a key or preferred aspect or embodiment. The word “or” is intended to be inclusive rather an exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggest otherwise.

The present disclosure relates to a peel and stick heating pad 100 for a breast of a user. The peel and stick heating pad 100 is particularly useful for women who need to heat their breast, such as for example, to assist in easing pain and/or soreness in their breast, purely as a warming device, or for any other applicable reason. Moreover, the peel and stick heating pad 100 may be useful for women with mastitis (breast inflammation). Further still, the peel and stick heating pad 100 may be used as a breast cancer therapy and may also used to ease the discomfort of engorgement as well just keeping the breast warm during cold days, such as during the winter.

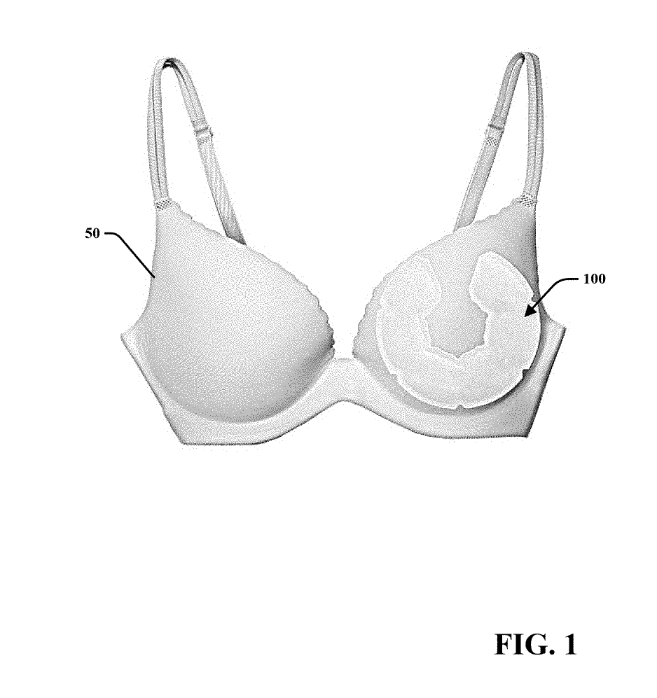

An embodiment of the present teachings is shown in FIG. 1. An exemplary embodiment of the peel and stick heating pad 100 is illustrated in FIG. 1. The peel and stick heating pad 100 may be adapted to selectively adhere to the inside of any bra 50, such as any bra available in the market. The peel and stick heating pad 100 does not need to be a specified configuration of a bra to work. Any available bra may work, including, without limitation, a strapless bra, strapped bra, a wired bra, etc. The peel and stick heating pad 100 may provide immediate and effective warming relief to help ease any kind of discomfort or pain, including the discomfort of engorgement. The peel and stick heating pad 100 may be used as a breast cancer therapy, with breast feeding, and mastitis (breast inflammation).

As shown in FIG. 1, the peel and stick heating pad 100 may be selectively attachable to the bra 50. Specifically, the peel and stick heating pad 100 may be selectively attached within the cup of the bra 50. The peel and stick heating pad 100 may have a profile such that when it is attached to the bra 50, it is virtually undetectable, i.e., a third party cannot see that peel and stick heating pad 100 of the wearer. Alternatively, the peel and stick heating pad 100 may be selectively attached to the breast of the user and then the bra can be worn over the peel and stick heating pad 100. The peel and stick heating pad 100 may include a cut-out shape such that the breast of the wearer fits generally around the peel and stick heating pad 100. By way of a non-limiting example, the peel and stick heating pad 100 may include 1 inch gaps straight through to generally circumscribe portions of the wearer's breast. This generally prevents a point or a cone from being formed, which allows the peel and stick heating pad 100 from being visible through a wearer's clothes and bra.

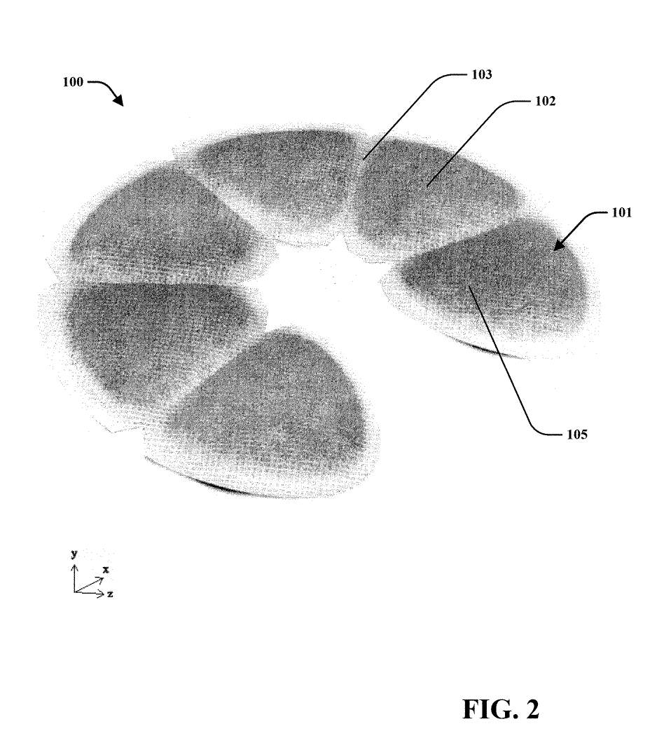

With respect to FIG. 2, the peel and stick heating pad 100 may include a plurality of pad members 101 generally positioned and configured to be adhered to the surface of the bra 50 (or directly to the user). As used herein, the word “plurality” means two or more. The plurality of pad members 101 may be a generally contoured shape that conforms to a variety of breast sizes and bra sizes or more specifically to a variety of cups and cup sizes. By way of a non-limiting example, the pad members 101 may be generally triangular or squares with a cut-out portion to prevent forming a cone shape on the wearer. The plurality of pad members 101 may generally abut one another and/or be spaced apart as shown in FIG. 2. The plurality of pad members 101 may be attached to form a generally U-shape for the peel and stick heating pad 100. The shape may be such that there is at least a one inch gap through the peel and stick heating pad 100. This may prevent a cone from being formed and may allow the peel and stick heating pad 100 from being seen through the bra and clothes of a wearer. Each of the plurality of pad members 101 may include a filling portion 102 and a fused portion 103. A heat-generating composition 105 may be located and filled in the filling portion 102 and separated by the fused portion 103 of the plurality of pad members 101. The heat generating composition 105 may be of any appropriate configuration. By way of a non-limiting example, the heat generating composition 105 may comprise iron powder, salt, water, an absorbent material, and activated carbon. When the pad members 101 are removed from their outer packaging, oxygen may drift across the pad members 101 permeable covering. With salt and water present, the oxygen reacts with the iron powder located inside to form iron oxide (Fe₂O₃) and release heat. The absorbent material within the heat generating composition 105 can be pulverized wood, a polymer such as polyacrylate, or a silicon-based mineral called vermiculite. It helps retain the moisture so that the reaction can occur. It should be understood, however, that this is merely an exemplary embodiment. Any appropriate configuration of heat generating composition 105 may be utilized. The present teachings are not limited to a specific configuration of such.

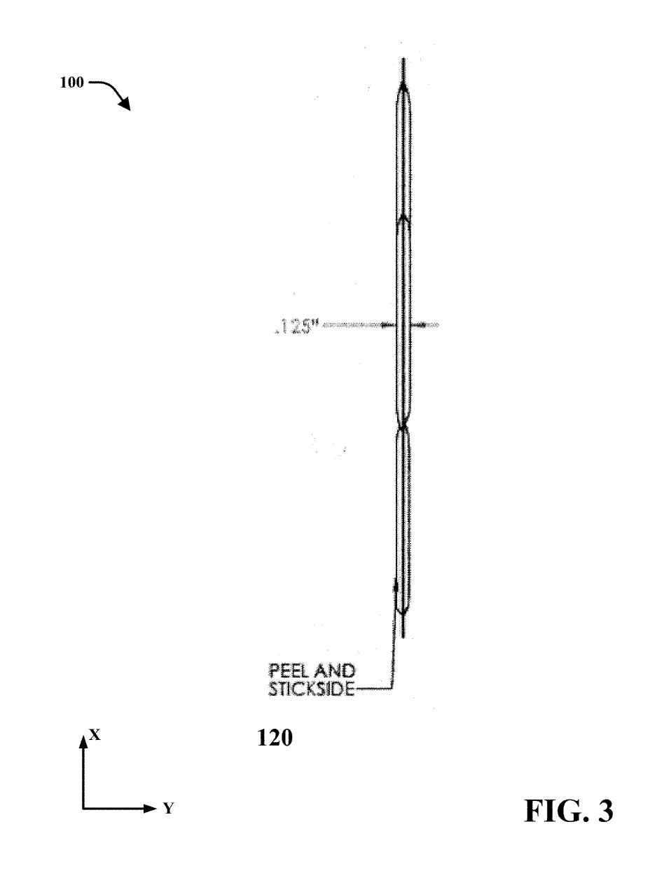

FIG. 3 illustrates a cross-sectional side view of an embodiment of the peel and stick heating pad 100. The peel and stick heating pad 100may be provided as a series of a uniform or varied (i.e. non-uniform) length or thickness. When provided as shapes of a substantial length, the shapes may be provided as uniform or random shapes and may be generally round, rectangular, hexagon, etc. In particular embodiment, the peel and stick heating pad 100 may have a thickness of from about 0.1 inch to about 0.15 inch, and in particular embodiment, about 0.125 inch. However, the thickness of the peel and stick heating pad 100 may be selected as desired for a particular application or intended purpose. For example, the thickness may be selected to make it virtually undetectable when worn by the user.

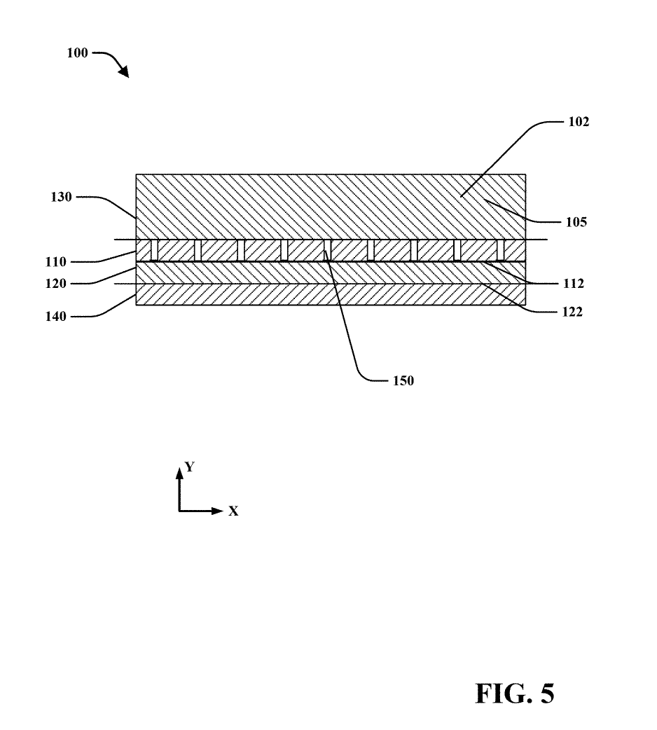

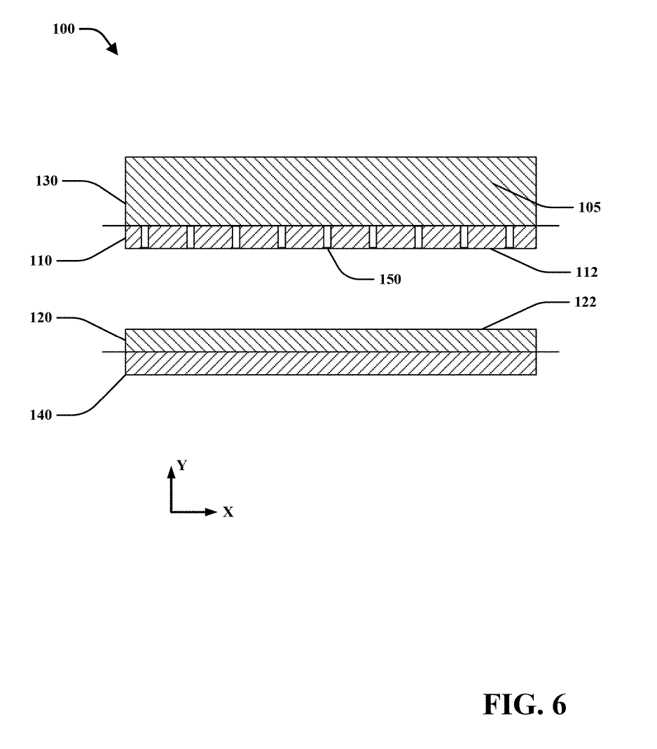

Referring to FIGS. 5 and 6, an embodiment of peel and stick heating pad 100 is shown. The peel and stick heating pad 100 may include a first pressure sensitive adhesive 110 having a first pressure sensitive adhesive surface 112, and a second pressure sensitive adhesive 120 having a second pressure sensitive adhesive 122. The first pressure sensitive adhesive 110 may be attached to a facestock layer 130. Similarly, the second pressure sensitive adhesives 120 may be attached to a liner layer 140. The surface 112 and/or the surface 122 may be modified with an additive or material to reduce the tack of the surface. The pressure sensitive adhesive surface 112 may be brought into contact with pressure sensitive adhesive surface 122, and, with the application of limited pressure, the surfaces will stick together to form a closure. The closure may be opened or peeled off by applying a pressure or force to pull the surfaces away from one another.

When the second pressure sensitive layer 120 and the liner layer 140 of one of the plurality of pad members 101 is selectively peeled off and removed from the first pressure sensitive adhesive 110, the first pressure sensitive adhesive 110 may be configured to adhere between the plurality of pad members 101 and an inside surface of the bra 50. In addition or alternatively, the first pressure sensitive adhesive 110 may adhere to the skin of the user. The plurality of pad members 101 may be of a slim profile so that the peel and stick heating pad 100 may be visually unnoticeable. Each of the plurality of pad members 101 may include the second pressure sensitive layer 120 and the liner layer 140 or only a select portion of the plurality of pad members 101 may include the second pressure sensitive layer 120.

In one embodiment, the first pressure sensitive adhesive 110 may be provided with a modified surface 112. In one embodiment, the second pressure sensitive adhesive 120 may be provided with a modified surface 122. In still another embodiment, both of the pressure sensitive adhesives 110 and 120 may be provided with modified surfaces 112, 122, respectively. Further, it will be appreciated that the respective adhesive components (e.g., 110 or 120) may be the same or different. For example, the adhesive components may use the same or different adhesive material. If both pressure sensitive adhesive surfaces are modified, the material providing the modified surface may be the same or different from one another.

The pressure sensitive adhesive employed as part of the peel and stick heating pad 100 is not particularly limited and may be chosen as desired for a particular purpose or intended application but then may be removed therefrom without damaging the bra or affecting the skin of the user. Useful pressure sensitive adhesives may include, for example, rubber-based, and acrylic-based adhesives. The adhesives can be solvent-based, water-based emulsions and suspensions, or hot melt. Additionally, the adhesives can be categorized as permanent, removable, repositionable or ultra-removable, as some non-limiting examples. The layer of pressure sensitive adhesive can be applied by any known method, including but not limited to, for example, slot coating, curtain coating, knife coating, gravure coating and extrusion coating. It should be understood that if a low-adhesion pressure sensitive adhesive is employed, for example, a removable, repositionable or ultra-removable adhesive, the release-coated liner might not require a release coating.

The liner layer 140 of the peel and stick heating pad 100 may be used with a “peel away” opening. A plurality of liner layer 140 may be utilized on a single layer. By way of a non-limiting example, two, three, four or more such liner layer 140 may be utilized in conjunction with a single liner layer. Any such configuration is contemplated hereby.

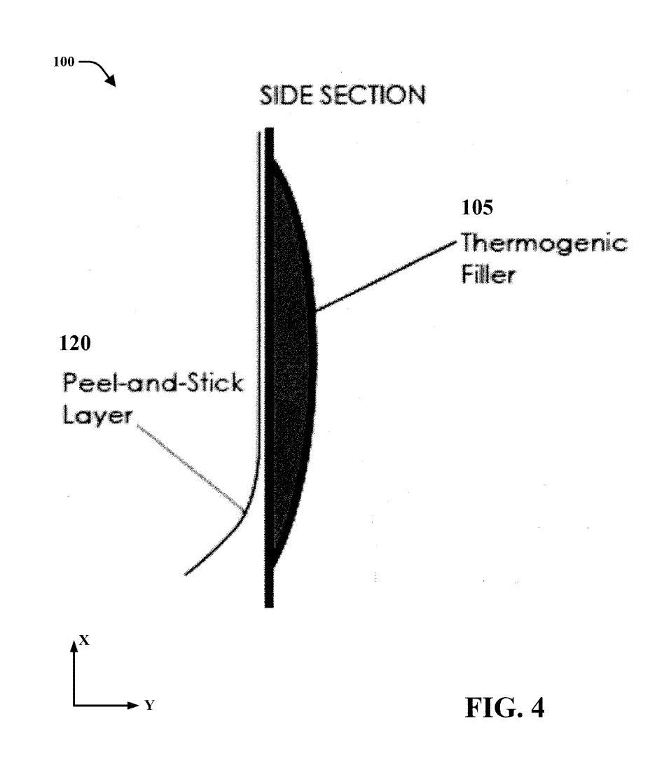

With respect to the FIG. 4, the heat-generating composition 105 of the peel and stick heating pad 100 for breast may be stored in and come from a chemical reaction. By way of a non-limiting example, the heater or warmers may operate on a chemical reaction with air similar to rusting. The warmer ingredients may include iron, water, cellulose, vermiculite, activated carbon and salt. The heating process may take place as follows:

The iron in the plurality of pad members 101, when exposed to oxygen, oxidizes and therefore produces heat (aka, “Air Activated”). The heat-generating composition 105 may be sealed by the second pressure sensitive adhesive 120 followed by the liner layer 140.

In the present disclosure, disposable chemical pads may employ a one-time exothermic chemical reaction. In one embodiment, the reaction may be triggered by unwrapping an air-tight packet containing slightly moist iron powder and salt or catalysts which rusts over a period of hours after being exposed to oxygen in the air through a pour 150.

The pour 150 may be provided in a random array or in a regular or uniform pattern. The generally porous material may be required to provide the pour 150 in a regular/uniform pattern. This allows a user to control the surface topography of the pressure sensitive adhesive. The pour 150 may be provided as discrete dots or shapes or may be provided as a series of filaments or fibrils of a uniform or varied (i.e. non-uniform) length or thickness.

When iron oxidizes it produces iron oxide, more commonly referred to as rust.

The heat-generating composition 105 within the plurality of pad members 101 may be a heat generating substance comprised of a mixture of an oxidizable metal—generally, but not limited to, iron powder, filings or shavings; powdered, activated carbon; water and salt. Other ingredients may include zinc, magnesium, or other metals, and the metals may be mixed with catalysts and assistants such as chloride ions and active carbon, as well as fillers and moisteners, such as bentonite and cellulose compounds, or any combination of other ingredients, which, when exposed to oxygen, create an exothermic chemical reaction.

Examples of suitable fillers include, but are not limited to, salt, carbon, vermiculite, etc.

The salt may act as a catalyst. The carbon may assist dispersing the heat uniformly. By way of non-limiting example, the vermiculite may be used as an insulator for the purpose of retaining the heat and cellulose may be added as a filler.

All of these ingredients may be surrounded by a bag, such as a polypropylene bag. The bag may be formed from any suitable material, including, without limitation any type of air-tight material. Polypropylene may allow air to permeate the ingredients while holding in moisture.

The absorbent material may be vermiculite, pulverized wood, or a superabsorbent polymer such as polyacrylate. It may assist retaining the moisture so that the reaction may occur. The activated carbon may assist to evenly disperse the heat produced, which may vary from about 125 degree F. to about 145 degree F. and particularly average about 135 degree F.

The heat may last for approximately 8 hours.

In other embodiments, the heat-generating composition 105 may comprise a material that is capable of being warmed through being microwaved. The material could be anything that can be microwaved that retains heat for a predetermined amount of time. Such material may include, without limitation, rice, dried corn, flaxseed or the like.

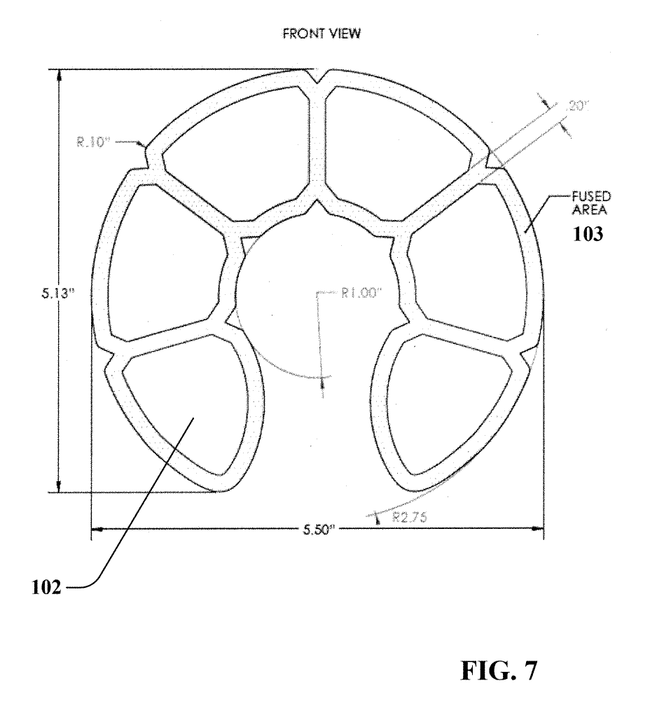

Referring to FIG. 7, the peel and stick heating pad 100 may include a generally contoured shape that conforms to a variety of breast sizes and bra sizes. By way of a non-limiting example, an inner diameter described herein may be about 1 inch—e.g., the gap between portions of the peel and stick heating pad 100 may be about an inch. The length of the inner diameter may be selected for the type of the plurality of pad members 101 and the desired size for the pad members 101. By way of a non-limiting example, an outer diameter described herein may be from about 4 to about 6 inches. The length of the outer diameter may be selected for the type of the plurality of pad members 101 and the desired size for the pad members 101. The present disclosure, however, is not limited to any particular length of the pad members 101.

The plurality of pad members 101 may also include a filling portion 102 and a fused portion 103. A plurality of pad members 101 may be separated by the fused portion 103. By way of a non-limiting example, a width of the fused portion 103 described herein may be from about 0.1 inch to about 0.3 inch. The width of the fused portion 103 may be selected for the type of the plurality of pad members 101 and the desired shape for the pad members 101. The present disclosure, however, is not limited to any particular width of the fused portion 103.

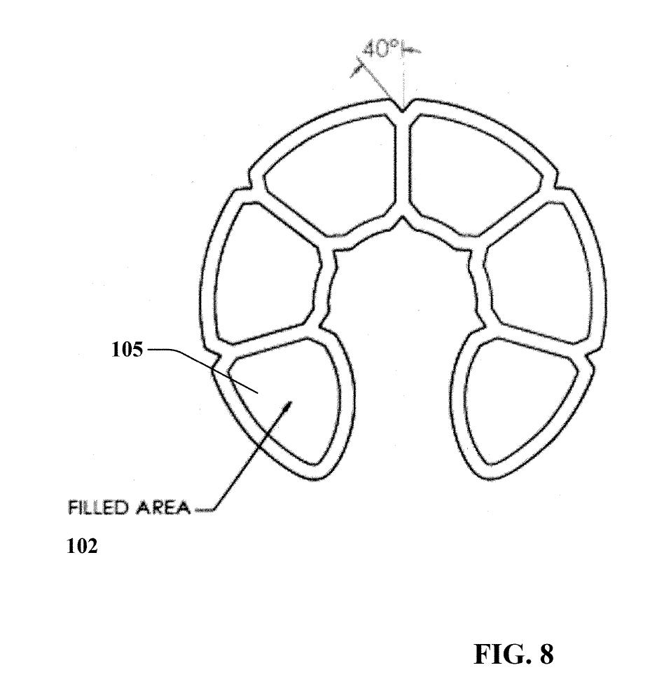

Referring to FIG. 8, each of the plurality of pad members 101 may include an angle. This angle may allow the width of the fused portion 103 be uniform across peel and stick heating pad 100. In this particular embodiment, the angle between the plurality of pad members 101 may be from about 30 to about 50 degree and particularly about 40 degree.

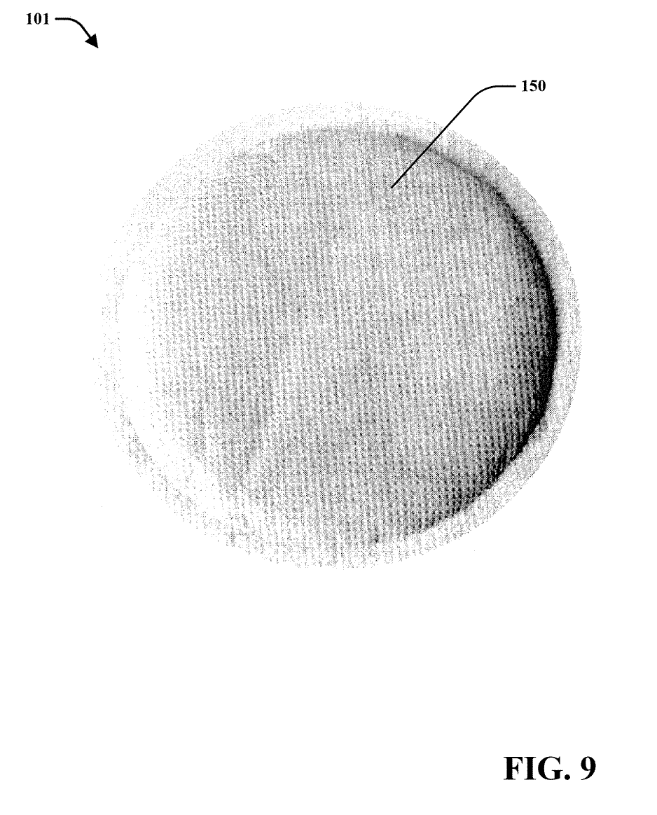

Additionally, an alternative embodiment is shown in FIG. 9 as an exemplary view of the plurality of pad members 101 after peeling off of the liner layer 120 that includes a generally round shape.

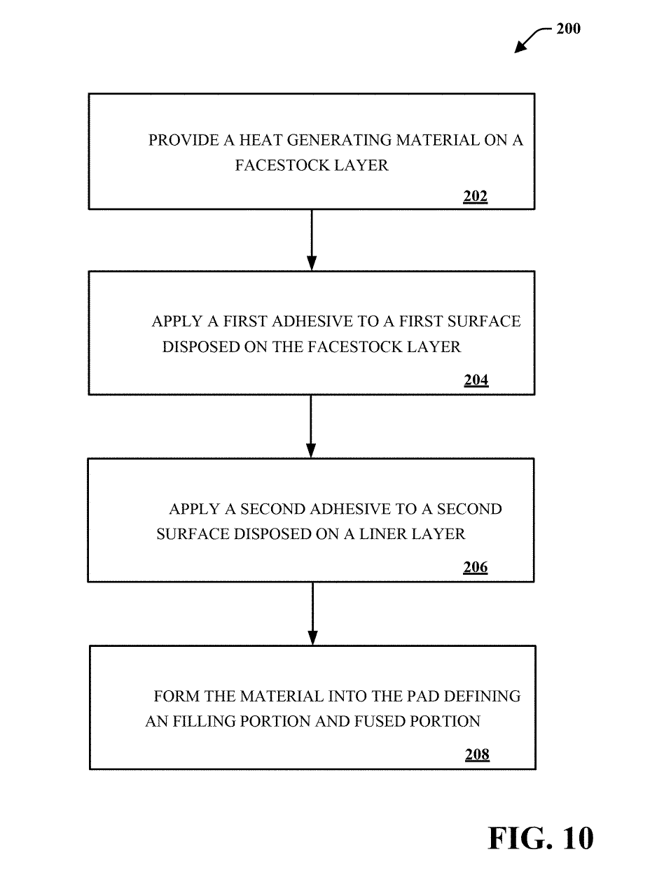

In view of the subject matter described herein, methods that may be related to various embodiments may be better appreciated with reference to the flowchart of FIG. 10. While the method 200 is shown and described as a series of blocks, it is noted that associated methods or processes are not limited by the order of the blocks. It is further noted that some blocks and corresponding actions may occur in different orders or concurrently with other blocks. Moreover, different blocks or actions may be utilized to implement the methods described hereinafter.

At 202, a method of manufacturing a pad may include providing a heat-generating material into the facestock layer. At 204, the method may also include applying a first pressure sensitive adhesive to a surface disposed on the facestock layer. At 206, a second pressure sensitive adhesive material may be applied to a surface disposed on the liner layer. At 208, the method further includes forming the material into the pad defining an enclosure, such as for example, filling portion and a fused portion.

Although the embodiments of the present invention have been illustrated in the accompanying drawings and described in the foregoing detailed description, it is to be understood that the present invention is not to be limited to just the embodiments disclosed, but that the invention described herein is capable of numerous rearrangements, modifications and substitutions without departing from the scope of the claims hereafter. By way of non-limiting example, other peel and stick heating pad for breast shapes may be utilized, different adhesives may be utilized, etc. The claims as follows are intended to include all modifications and alterations insofar as they come within the scope of the claims or the equivalent thereof.

Having thus described the invention, we claim:

1. A pad comprising:
a facestock layer having a plurality of pad members, each of the plurality of pad members capable of being selectively activated, the each of the plurality of pad members comprising a heat-generating composition generating a heat to a temperature;
a first pressure sensitive adhesive layer coupled to the facestock, the first pressure sensitive adhesive layer including a porous material configured to control exposure to air;
a second pressure sensitive adhesive layer having an upper adhesive surface, wherein the adhesive surfaces of the first and second pressure sensitive adhesives are releasably securable to one another to form a closure; and
a liner layer configured to be adhere to the second pressure sensitive layer.

2. The pad of claim 1, wherein the temperature is about 125 to about 145 degrees F.

3. The pad of claim 1, wherein the facestock layer contains an oxidizable substance.

4. The pad of claim 1, wherein the facestock layer contains two or more substances, the two or more substances producing a heat-generating reaction when exposed to an air.

5. The pad of claim 4, wherein the pad is disposable after producing one heat-generating reaction.

6. The pad of claim 1, further comprising a bra wherein the pad is selectively attached thereto.

7. The pad of claim 6, wherein the plurality of pad members forms a generally U-shape.

8. The pad of claim 7, wherein the pad is attached within a cup of the bra.

9. The pad of claim 8, wherein the pad has a profile conforms to the cup of the bra.

10. A method of forming an article having a heating pad for breast, the method comprising;
providing a heat-generating material;
applying a first adhesive to a surface disposed on a first surface and a second adhesive to a second surface of the pad;
forming the heat-generating material into the pad.

11. The method of claim 10, wherein forming the heat-generating material into the pad further comprises forming a plurality of pad members with a generally U-shape capable of fitting within a cup of a bra.
